(19) United States
(12) Patent Application Publication
(10) Pub. No.: US 2025/0297997 A1
(43) Pub. Date: Sep. 25, 2025

(54) CONTINUOUS MONITORING DEVICE FOR DIRECT CARBON EMISSION OF COMPLETE SET OF CHEMICAL EQUIPMENT

(71) Applicant: China Special Equipment Inspection & Research Institute, Beijing (CN)

(72) Inventors: Sheng Chen, Beijing (CN); Yue YU, Beijing (CN); Zhiyuan Han, Beijing (CN)

(21) Appl. No.: 18/771,985

(22) Filed: Jul. 12, 2024

(30) Foreign Application Priority Data
Mar. 20, 2024 (CN) 202410323564.2

(51) Int. Cl. G01N 33/00; B01D 45/02

(52) U.S. Cl. CPC G01N 33/0027; B01D 45/02

(57) ABSTRACT

A continuous monitoring device for direct carbon emission of a complete set of chemical equipment is provided, relating to the field of carbon emission monitoring. A measuring pipeline module in the device includes a sampling gas measuring pipeline, an auxiliary gas measuring pipeline, an air measuring pipeline, and a tail gas measuring pipeline. The measuring pipeline module is configured to adjust a degree of opening of each measuring pipeline to measure different types of sampling gases. A gas treatment module can achieve sufficient and efficient combustion. Data collection, processing and control modules can collect temperatures, pressures, flow rates and concentration data of different pipelines, so as to determine data validity and calculate a carbon emission concentration in real time, and automatically regulate abnormal working conditions or remotely regulate abnormal working conditions by experts.

### TECHNICAL FIELD

The present disclosure relates to the field of carbon emission monitoring, and in particular to a continuous monitoring device for direct carbon emission of a complete set of chemical equipment.

### BACKGROUND

Carbon emission source monitoring is the most direct way of carbon accounting and carbon emission reduction effect evaluation in production process, which is closely related to industries and production processes. Carbon emission forms in different industries and production processes are complex (point source emission and fugitive emission, among which there are many types of point source emission, such as direct emission from process emptying pipes and chimneys, and emission after torch treatment) and have great differences (concentrations, flow rates and types of emission gases, location arrangement, monitoring methods, etc.), which makes the existing carbon monitoring difficult and the laws, standards and technical specifications immature.

At present, the electric power, building material and steel industries have carried out the greenhouse gas emission monitoring pilot on the basis of the existing waste gas continuous automatic monitoring system and compared monitoring results with the accounting results. According to the statistics of carbon emissions in China in 2020, in addition to the electric power, building material and steel industries, the chemical industry has more carbon emissions, second only to the steel industry. The total carbon emission of chemical industry in 2020 is about 1 billion tons, including 350 million tons from petrochemical, 540 million tons from coalification, and 100 million tons from natural gas chemical and others. Therefore, the carbon emission in chemical industry cannot be ignored. However, the carbon monitoring in the chemical industry is still blank at present.

The complete set of chemical equipment has large carbon emission, high concentration and many forms. Different from the traditional single stationary carbon emission source monitoring, the common carbon monitoring technology based on gas types (CO, CO2) is difficult to be applied to different carbon emission forms. The process emissions of various pressure-bearing equipment, such as direct emptying through process pipelines such as Rectisol, torch combustion emptying, reactor tail gas and process waste gas scrubbing device treatment emptying, account for more than 63.5% of the total carbon emission. There are great differences in gas types, contents, flow rates and installation measurement modes under various emission forms of the complete set of chemical equipment, so it is impossible to directly apply the existing greenhouse gas carbon emission technologies of boiler flue gas in China and at abroad. For example, the gas types include greenhouse gases such as methane, carbon monoxide, carbon dioxide, nitrous oxide, and many VOCs such as alkanes and olefins; the gas content is that CO2 content in an emptying pipeline of coal gasification process is as high as 80% vol or more; the gas flow rate is that different from large-flow discharge of the boiler, low-flow discharge below 1000 m3/h is the majority.

Therefore, it is necessary to develop an accurate, second-level, long-term stable and continuous monitoring device and method for direct carbon emission, which are not limited by the types and contents of gases measured in the complete set of chemical equipment.

### SUMMARY

An objective of the present disclosure is to provide a continuous monitoring device for direct carbon emission of a complete set of chemical equipment, which can achieve accurate, second-level, long-term stable and continuous monitoring of direct carbon emission without being limited by the types and contents of gases measured in complete set of chemical equipment.

To achieve the objective above, the present disclosure employs the following technical solution:
A continuous monitoring device for direct carbon emission of a complete set of chemical equipment includes a measuring pipeline module, a gas mixer, a gas treatment module, a data collection module, a data processing module, and a data control module.

The measuring pipeline module includes a sampling gas measuring pipeline, an auxiliary gas measuring pipeline, an air measuring pipeline, and a tail gas measuring pipeline. The measuring pipeline module is configured to adjust a degree of opening of each measuring pipelines to measure different types of sampling gases.

The sampling gas measuring pipeline, the auxiliary gas measuring pipeline and the air measuring pipeline all communicate with the gas treatment module through the gas mixer, and the tail gas measuring pipeline communicates with the gas treatment module.

The gas treatment module is used for gas combustion, cooling, and gas-liquid separation.

The data collection module is connected to the measuring pipeline module, and configured to collect temperatures, pressures, flow rates, gas concentration data of different measuring pipelines of the measuring pipeline module and flame conditions.

The data processing module is connected to the data collection module and the data control module, and configured to determine data validity and calculate carbon emission concentration in real time according to the temperatures, the pressures, the flow rates, gas concentration data of the different measuring pipelines and flame conditions.

The data control module is further connected to the measuring pipeline module, and configured to perform an abnormal working condition regulation or a remote expert regulation according to a determination result and a calculation result.

Alternatively, the sampling gas measuring pipeline includes a regulating valve, a filter, an air compressor pump, a buffer tank, a pressure sensor, a temperature sensor, a flowmeter, a CH4 concentration detector, a CO2 concentration detector, a N2O concentration detector, and a flame arrester connected in sequence.

When a sampling gas is directly discharged to atmosphere without combustion in a process, the sampling gas is connected to tail treatment device through the flame arrester, and a check valve, or directly discharged to the atmosphere.

When the sampling gas needs to be burned and then discharged to the atmosphere in the process, the sampling gas is connected to the gas mixer to be burned and discharged to the atmosphere in the process. Meanwhile, the N2O concentration detector is no longer arranged in the sampling gas measuring pipeline.

Alternatively, the auxiliary gas measuring pipeline includes a regulating valve, a buffer tank, a pressure sensor, a temperature sensor, a flowmeter, a CH4 concentration detector, a CO2 concentration detector, a CO concentration detector, and a flame arrester connected in sequence.

Alternatively, the air measuring pipeline includes a regulating valve, a filter, an air compressor pump, a buffer tank, a pressure sensor, a temperature sensor, a flowmeter, and a CO2 concentration detector connected in sequence.

Alternatively, the tail gas measuring pipeline comprises a filter, an air compressor pump, a buffer tank, a pressure sensor, a temperature sensor, a flowmeter, and a CO2 concentration detector connected in sequence.

Alternatively, the gas treatment module includes a flameless burner, a combustion furnace, a condenser, and a gas-liquid separator.

The flameless burner is arranged in the combustion furnace.

An automatic drainage port is arranged at a bottom of the combustion furnace, and a safety valve and a bursting disc are arranged at a top of the combustion furnace.

The condenser and the combustion furnace are connected side by side, and the condenser is configured to regulate a flow rate of condensed water according to a measured tail gas temperature to control a temperature.

The gas-liquid separator employs a vertical over-entering and down-out structure, and an outlet pipe at a bottom of the gas-liquid separator employs a liquid seal form.

Alternatively, a water-cooling coil is arranged in the buffer tank.

Alternatively, the data processing module is configured to compare CO2 mass flow rates respectively measured by the sampling gas measuring pipeline, the auxiliary gas measuring pipeline and the air measuring pipeline, a sum of the CO2 mass flow rates respectively measured by the sampling gas measuring pipeline, the auxiliary gas measuring pipeline and the air measuring pipeline with a CO2 mass flow rate determined by the tail gas measuring pipeline, so as to determine the data validity.

The data processing module is configured to convert a volume flow rate into a mass flow rate based on an ideal gas law, and to account the carbon emission concentration in real time according to agas type.

According to specific embodiments of the present disclosure, the present disclosure has the following technical effects:

A continuous monitoring device for direct carbon emission of a complete set of chemical equipment is provided, relating to the field of carbon emission monitoring. A measuring pipeline module in the device includes a sampling gas measuring pipeline, an auxiliary gas measuring pipeline, an air measuring pipeline, and a tail gas measuring pipeline. The measuring pipeline module is configured to adjust a degree of opening of each measuring pipeline to measure different types of sampling gases. A gas treatment module can achieve sufficient and efficient combustion. Data collection, processing and control modules can collect temperatures, pressures, flow rates and concentration data of different pipelines, so as to determine data validity and calculate a carbon emission concentration in real time, and automatically regulate abnormal working conditions or remotely regulate abnormal working conditions by experts. The problems of low monitoring accuracy of carbon emission, uncertain type and content of sampling gas, large pressure and temperature fluctuation and poor long-term stability can be solved. Therefore, accurate, second-level, long-term stable and continuous monitoring of direct carbon emission can be achieved without being limited by types and contents of gases measured in the complete set of chemical equipment.

### BRIEF DESCRIPTION OF THE DRAWINGS

To describe the technical solutions of the embodiments of the present disclosure or in the prior art more clearly, the following briefly introduces the accompanying drawing required for describing the embodiments. Apparently, the accompanying drawing in the following description shows merely some embodiments of the present disclosure, and those of ordinary skill in the art may still derive other drawings from the accompanying drawing without creative efforts.

FIG. 1 is a structural schematic diagram of a continuous monitoring device for direct carbon emission of a complete set of chemical equipment according to the present disclosure.

In the drawing:
1—sampling gas; 2—auxiliary gas; 3—air; 4—tail gas; 5—regulating valve; 6—filter; 7—air compressor pump; 8—gas buffer tank; 9—pressure sensor; 10—temperature sensor; 11—flowmeter; 12—CH4 concentration detector; 13—CO2 concentration detector; 14—N2O concentration detector; 15—CO concentration detector; 16—flame arrester; 17—check valve; 18—gas mixer; 19—flameless burner; 20—combustion furnace; 21—condenser; 22—gas-liquid separator; 23—tail gas treatment; 24—wastewater treatment.

### DETAILED DESCRIPTION OF THE EMBODIMENTS

The following clearly and completely describes the technical solutions in the embodiments of the present disclosure with reference to the accompanying drawing in the embodiments of the present disclosure. Apparently, the described embodiments are merely a part rather than all of the embodiments of the present disclosure. All other embodiments obtained by a person of ordinary skill in the art based on the embodiments of the present disclosure without creative efforts shall fall within the scope of protection of the present disclosure.

An objective of the present disclosure is to provide a continuous monitoring device for direct carbon emission of a complete set of chemical equipment, which can achieve accurate, second-level, long-term stable and continuous monitoring of direct carbon emission without being limited by the types and contents of gases measured in complete set of chemical equipment.

In order to make the above objectives, features and advantages of the present disclosure more clearly, the present disclosure is further described in detail below with reference to the accompanying drawing and specific embodiments.

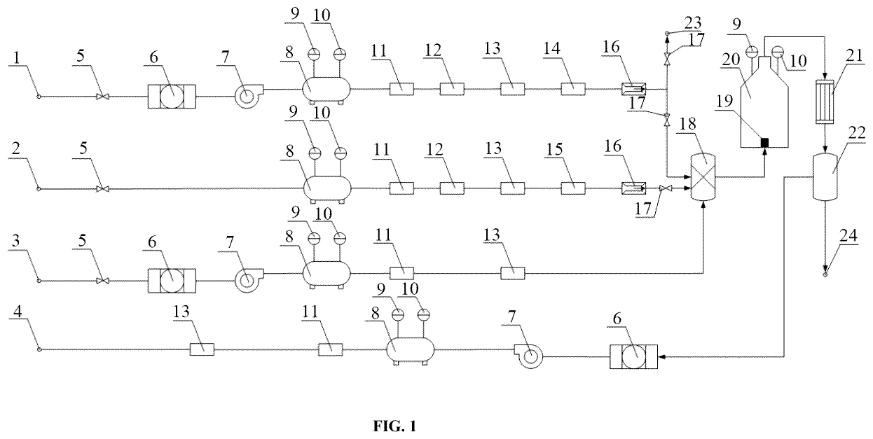

As shown in FIG. 1, a continuous monitoring device for direct carbon emission of a complete set of chemical equipment provided by the present disclosure includes a measuring pipeline module, a gas mixer 18, a gas treatment module, a data collection module, a data processing module, and a data control module.

The measuring pipeline module includes a sampling gas 1 measuring pipeline, an auxiliary gas 2 measuring pipeline, an air 3 measuring pipeline, and a tail gas 4 measuring pipeline. The measuring pipeline module is configured to adjust degrees of opening of various measuring pipelines to measure different types of sampling gases 1.

The sampling gas 1 measuring pipeline, the auxiliary gas 2 measuring pipeline and the air 3 measuring pipeline all communicate with the gas treatment module through the gas mixer 18, and the tail gas 4 measuring pipeline communicates with the gas treatment module.

The gas treatment module is used for gas combustion, cooling, and gas-liquid separation.

The data collection module is connected to the measuring pipeline module, and configured to collect temperatures, pressures, flow rates, gas concentration data and flame conditions of different measuring pipelines of the measuring pipeline module.

The data processing module is connected to the data collection module and the data control module, and configured to determine data validity and calculate a carbon emission concentration in real time according to the temperatures, pressures, flow rates, gas concentration data of different measuring pipelines and a flame condition.

The data control module is further connected to the measuring pipeline module, and configured to perform abnormal working condition regulation or remote expert regulation according to a determination result and a calculation result.

The abnormal working conditions mainly include four phenomenas: overpressure, over-temperature, burner flameout, and insufficient combustion. If a working pressure exceeds the maximum design pressure by 1.05-1.5 times, the overpressure relief is carried out. If a working temperature exceeds the design temperature by 1.05-1.5 times, the circulating water temperature is automatically reduced or the circulating water flow is increased in proportion. The burner flameout can be regulated by automatic ignition of the burner, flowrate regulation of the auxiliary gas 2 within 10%, or remote expert diagnosis. If the combustion is insufficient, the flow rate of the auxiliary gas 2 measuring pipeline can be adjusted through the temperature feedback. According to remote expert regulation, the causes of abnormal working conditions can be determined according to the collected data, and an operation status of the device can be controlled by regulating each measuring pipeline and the circulating quantity of the cooling water.

The sampling gas 1 measuring pipeline includes a regulating valve 5, a filter 6, an air compressor pump 7, a buffer tank 8, a pressure sensor 9, a temperature sensor 10, a flowmeter 11, a CH4 concentration detector 12, a CO2 concentration detector 13, a N2O concentration detector 14, and a flame arrester 16, connected in sequence. The CH4 concentration detector 12, the CO2 concentration detector 13 and the N2O concentration detector 14 are based on the principle of NDIR (Non-dispersive infrared) or TDLAS (Tunable Diode Laser Spectroscopy), and a volume fraction of a measured gas in a known closed optical path is calculated according to the Beer-Lambert law, and a sampling time is less than or equal to 5 s. In order to suppress signal attenuation of a detector, eliminate environmental interference, and improve accuracy and precision of detection, it is suggested that a concentration detector should be designed in a measurement and reference double-channel detection way. A measurement channel is configured to measure a concentration of a gas to be measured, and a reference channel signal is configured to measure concentrations of components in the environment, which has nothing to do with a concentration of the measured gas.

When the sampling gas 1 is directly discharged to atmosphere without combustion in a sampling gas 1 process, the sampling gas is connected to a tail gas treatment 23 device through the flame arrester 16 and a check valve 17, or directly discharged to the atmosphere.

When the sampling gas 1 needs to be burned and then discharged to the atmosphere in the sampling gas 1 process, the sampling gas 1 needs to be connected to the gas mixer 18 to be combusted and discharged to the atmosphere. Meanwhile, the N2O concentration detector 14 is no longer arranged in the sampling gas 1 measuring pipeline.

The auxiliary gas 2 measuring pipeline includes a regulating valve 5, a buffer tank, a pressure sensor 9, a temperature sensor 10, a flowmeter 11, a CH4 concentration detector 12, a CO2 concentration detector 13, a CO concentration detector 15, and a flame arrester 16 connected in sequence.

The auxiliary gas 2 is natural gas, fuel gas, CH4 or non-carbon combustion-supporting gas (such as hydrogen); the CO, CO2 and CH4 concentration detectors 15, 13, 12 can be increased or decreased according to gas types and content, but when the auxiliary gas 2 contains CH4, the CH4 concentration detector 12 must be kept. In the process, the sampling gas 1 is directly discharged to the atmosphere and can be directly burned, and then the auxiliary gas 2 measuring pipeline can be closed.

The air 3 measuring pipeline includes a regulating valve 5, a filter 6, an air compressor pump 7, a buffer tank, a pressure sensor 9, a temperature sensor 10, a flowmeter 11 and a CO2 concentration detector 13 connected in sequence.

The tail gas 4 measuring pipeline includes a filter 6, an air compressor pump 7, a buffer tank, a pressure sensor 9, a temperature sensor 10, a flowmeter 11 and a CO2 concentration detector 13 connected in sequence.

The gas treatment module includes a flameless burner 19, a combustion furnace 20, a condenser 21, and a gas-liquid separator 22.

The flameless burner 19 is arranged in the combustion furnace 20. A combustion head of the flameless burner 19 has three states: blue flame, red flame and red-blue flame, preferably red flame. The combustion adopts oxygen-excess combustion, and the flameless burner is provided with a flameout automatic igniter.

An automatic drainage port is arranged at a bottom of the combustion furnace 20 to prevent condensed water from gathering. A safety valve and a bursting disc are arranged at a top of the combustion furnace 20.

The condenser 21 and the combustion furnace 20 are connected side by side to prevent the condensed water from flowing back into the combustion furnace 20. The condenser 21 is configured to regulate a flow rate of condensed water according to a measured tail gas 4 temperature to control the temperature.

The gas-liquid separator 22 employs a vertical over-entering and down-out structure, and an outlet pipe at a bottom of the gas-liquid separator 22 employs a liquid seal form to prevent gas from stringing out.

The sampling gas 1, the auxiliary gas 2 and the air 3, after entering into the gas mixer 18 through measuring pipelines, respectively, are sent to the combustion furnace 20 for flameless combustion, and the tail gas 4 after combustion enters the tail gas 4 measuring pipeline for measurement. Different types of sampling gases 1 can be measured by adjusting degrees of opening of the test pipelines, so as to achieve accurate, second-level, long-term stable and continuous monitoring of direct carbon emission. The measuring pipeline employs a series combination design of a regulating valve 5, a filter 6, an air pressure pump 7, a buffer tank, a temperature sensor and a pressure sensor to achieve voltage stabilization, temperature stabilization, flow stabilization, drying and dust removal. The measuring pipeline employs a series combination design of a flowmeter 11 and an optical concentration detector to achieve quantitative measurement of volume fraction of each key component. The auxiliary gas 2 measuring pipeline employs combustion temperature parameter feedback to adjust the flow rate to achieve combustion state regulation. The combustion structure employs the design of the flameless burner 19 to achieve sufficient and efficient combustion. The condensation and separation structure employs a combined design of a water-cooling condenser 21 and the gas-liquid separator 22 to achieve cooling and gas-liquid separation. The data collection, processing and control modules are configured to collect temperatures, pressures, flow rates and concentration data of different pipelines, so as to determine data validity and calculate a carbon emission concentration in real time, and automatically regulate abnormal working condition or remotely regulate abnormal working condition by experts. The measuring pipelines and each module shall be made of austenitic stainless steel, and it is recommended to use materials such as 304L, 316L and 327H to prevent dew point and acid water corrosion.

A water-cooling coil is arranged in the buffer tank, which may be spiral or tube-bundle, and the temperature can be controlled within 80° C. by adjusting the flow rate of the condensed water according to the temperature of the measured gas.

The flowmeter 11 may be a Coriolis mass flowmeter 11, a restriction orifice type mass flowmeter 11 or a volume flowmeter 11. The flowmeter 11 for each of the sampling gas measuring pipeline of the auxiliary gas 1 measuring pipeline, auxiliary gas 2 measuring pipeline, and the air 3 measuring pipeline needs a flow restrictor, and the flow-restriction deviation is less than +/−5% of the actual value.

Data collected by the data collection module include the temperatures, pressures, flow rates, concentrations and flame conditions, and 4G/5G/Internet of Things is used for remote data transmission, and the data is transmitted to a display terminal for storage.

The data processing module is configured to compare CO2 mass flow rates respectively measured by the sampling gas 1 measuring pipeline, the auxiliary gas 2 measuring pipeline and the air 3 measuring pipelines and a sum of the CO2 mass flow rates respectively measured by the sampling gas 1 measuring pipeline, the auxiliary gas 2 measuring pipeline and the air 3 measuring pipelines with a CO2 mass flow rate determined by the tail gas 4 measuring pipeline, so as to determine data validity.

The data processing module is configured to convert a volume flow rate into a mass flow rate based on the ideal gas law, and to account the carbon emission concentration in real time according to the CO2, CH4, CO and N2O gases before and after combustion. The ideal gas law is as follows:
PV=nRT, P is pressure, pa; V is volume, m3; N is molar weight, mol; R is a gas constant of 8.314 J/(mol·K); and T is temperature, k.

The sampling gas 1 is directly discharged to the atmosphere in the process, and the mass flow rate of carbon emission is only calculated according to the data of the CO2, N2O and CH4 concentration detectors 13, 14, 12 in the sampling gas 1 measuring pipeline. The sampling gas 1 is discharged to the atmosphere after being burned in the process, such as a torch gas, which needs to be calculated according to the data of CO, CO2, N2O and CH4 concentration detectors 15, 13, 14, 12 in the sampling gas 1 measuring pipeline, the auxiliary gas 2 measuring pipeline, the air 3 measuring pipeline and the tail gas 4 measuring pipeline.

The technical features of the above embodiments can be combined at will. In order to make the description concise, not all possible combinations of the technical features in the above embodiments are described. However, it should be considered that these combinations of technical features fall within the scope recorded in this specification provided that these combinations of technical features do not have any conflict.

Specific examples are used herein for illustration of the principles and embodiments of the present disclosure. The description of the embodiments is merely configured to help illustrate the method and its core principles of the present disclosure. In addition, those of ordinary skill in the art can make changes in terms of specific embodiments and scope of application in accordance with the idea of the present disclosure. In conclusion, the content of this specification shall not be construed as a limitation to the present disclosure.

What is claimed is:

1. A continuous monitoring device for direct carbon emission of a complete set of chemical equipment, comprising a measuring pipeline module, a gas mixer, a gas treatment module, a data collection module, a data processing module and a data control module;
wherein the measuring pipeline module comprises a sampling gas measuring pipeline, an auxiliary gas measuring pipeline, an air measuring pipeline, and a tail gas measuring pipeline; the measuring pipeline module is configured to adjust a degree of opening of each measuring pipeline to measure different types of sampling gases;
the sampling gas measuring pipeline, the auxiliary gas measuring pipeline and the air measuring pipeline all communicate with the gas treatment module through the gas mixer, and the tail gas measuring pipeline communicates with the gas treatment module;
the gas treatment module is used for gas combustion, cooling, and gas-liquid separation;
the data collection module is connected to the measuring pipeline module, and configured to collect temperatures, pressures, flow rates, gas concentration data of different measuring pipelines of the measuring pipeline module and flame conditions;
the data processing module is connected to the data collection module and the data control module, and configured to determine data validity and calculate carbon emission concentration in real time according to the temperatures, the pressures, the flow rates, the gas concentration data of the different measuring pipelines and the flame conditions; and
the data control module is further connected to the measuring pipeline module, and configured to perform an abnormal working condition regulation or a remote expert regulation according to a determination result and a calculation result.

2. The continuous monitoring device for direct carbon emission of the complete set of chemical equipment according to claim 1, wherein the sampling gas measuring pipeline comprises a regulating valve, a filter, an air compressor pump, a buffer tank, a pressure sensor, a temperature sensor, a flowmeter, a CH4 concentration detector, a CO2 concentration detector, a N2O concentration detector and a flame arrester connected in sequence;
when a sampling gas is directly discharged to atmosphere without combustion in a process, the sampling gas is connected to the tail treatment device through the flame arrester and a check valve, or directly discharged to the atmosphere;
when the sampling gas needs to be burned and then discharged to the atmosphere in the process, the sampling gas is connected to the gas mixer to be burned and discharged to the atmosphere in the process; and meanwhile, the N2O concentration detector is no longer arranged in the sampling gas measuring pipeline.

3. The continuous monitoring device for direct carbon emission of the complete set of chemical equipment according to claim 1, wherein the auxiliary gas measuring pipeline comprises a regulating valve, a buffer tank, a pressure sensor, a temperature sensor, a flowmeter, a CH4 concentration detector, a CO2 concentration detector, a CO concentration detector and a flame arrester connected in sequence.

4. The continuous monitoring device for direct carbon emission of the complete set of chemical equipment according to claim 1, wherein the air measuring pipeline comprises a regulating valve, a filter, an air compressor pump, a buffer tank, a pressure sensor, a temperature sensor, a flowmeter and a CO2 concentration detector connected in sequence.

5. The continuous monitoring device for direct carbon emission of the complete set of chemical equipment according to claim 1, wherein the tail gas measuring pipeline comprises a filter, an air compressor pump, a buffer tank, a pressure sensor, a temperature sensor, a flowmeter and a CO2 concentration detector connected in sequence.

6. The continuous monitoring device for direct carbon emission of the complete set of chemical equipment according to claim 1, wherein the gas treatment module comprises a flameless burner, a combustion furnace, a condenser, and a gas-liquid separator;
the flameless burner is arranged in the combustion furnace;
an automatic drainage port is arranged at a bottom of the combustion furnace, and a safety valve and a bursting disc are arranged at a top of the combustion furnace;
the condenser and the combustion furnace are connected side by side, and the condenser is configured to regulate a flow rate of condensed water according to a measured tail gas temperature to control a temperature; and
the gas-liquid separator employs a vertical over-entering and down-out structure, and an outlet pipe at a bottom of the gas-liquid separator employs a liquid seal form.

7. The continuous monitoring device for direct carbon emission of the complete set of chemical equipment according to claim 1, wherein a water-cooling coil is arranged in the buffer tank.

8. The continuous monitoring device for direct carbon emission of the complete set of chemical equipment according to claim 1, wherein the data processing module is configured to compare CO2 mass flow rates respectively measured by the sampling gas measuring pipeline, the auxiliary gas measuring pipeline and the air measuring pipeline, a sum of the CO2 mass flow rates respectively measured by the sampling gas measuring pipeline, the auxiliary gas measuring pipeline and the air measuring pipeline with a CO2 mass flow rate determined by the tail gas measuring pipeline, so as to determine the data validity; and
the data processing module is configured to convert a volume flow rate into a mass flow rate based on an ideal gas law, and to account the carbon emission concentration in real time according to a gas type.
